(19) United States
(12) Patent Application Publication
(10) Pub. No.: US 2023/0398270 A1
(43) Pub. Date: Dec. 14, 2023

(54) BREAST PUMP INSERT

(71) Applicant: Milkdrop IP Pty Ltd, Bright (AU)

(72) Inventors: Alexandra SINICKAS, Bright, Victoria (AU); Andrew MASON, Bright, Victoria (AU); Daniel PROHASKY, Victoria (AU); Ravi BESSABAVA, Bright, Victoria (AU)

(73) Assignee: Milkdrop IP Pty Ltd, Bright (AU)

(21) Appl. No.: 18/247,109

(22) PCT Filed: Sep. 29, 2021
(86) PCT No.: PCT/AU2021/051131

§ 371 Date: Mar. 29, 2023

(30) Foreign Application Priority Data
Sep. 29, 2020 (AU) 2020903515

(51) Int. Cl. A61M 1/06

(52) U.S. Cl. CPC A61M 1/066

(57) ABSTRACT

A breast pump funnel integral or engageable with a breast pump providing intermittent pumping for drawing breast milk into a connected container, the breast pump funnel having: an outer frame and an inner malleable liner located at least partially within the outer frame and, in use, forming a contact surface to the breast. The outer frame, the inner malleable liner or both form a continuous restricting funnel for receiving an intermittent negative pressure pumping.

### FIELD OF THE INVENTION

The present invention relates to a breast pump insert for use in a breast pump to aid expression of breastmilk.

In particular the present invention relates to a breast pump insert that can fit a variety of breast pumps and aid efficiency of milk production and substantially reduces pain and discomfort of mothers.

The invention has been developed primarily for use in/with a breast pump for aid expression of breastmilk and will be described hereinafter with reference to this application. However, it will be appreciated that the invention is not limited to this particular field of use.

### BACKGROUND OF THE INVENTION

Breast pumps generally comprise a vacuum pump, a breast cap having a funnel portion and a tube portion, the breast cap being connected to the pump and placed over the areola of a human mother's breast, and a collecting container connected to the breast cap for collecting milk that is pumped off.

Breast pumps are used by mothers to express milk for a number of reasons including for example: if they are away from home (e.g. for work), if the baby has difficulty sucking because they are premature, have a tongue tie or other issues, if the mum is sick and can't be with the baby, if the mum needs to increase her milk supply, or re-lactate.

Typically, new mothers experience some kind of discomfort or pain when using breast pumps to collect breast milk. This is due to outdated and over-simplified lactation pump fixtures. There are three problems experienced by mothers:
1. Pumping can be painful and uncomfortable for mothers, particularly when the breast is full and sensitive. Some mothers have had the skin on their areolas rubbed raw or had bleeding of the nipple due to the nipple and areola being repeatedly and forcefully sucked up the tube portion of the breast cap. After pumping, the nipple of some mums is swollen and sore, which is not what is experienced when a baby suckles. Research has found that 65% of mothers suffer nipple damage, pain or discomfort when using breast pumps. Some draw blood and others have bruising and swelling. This can lead to mastitis and infection;
2. Pumping doesn't collect as much milk as it could. Mothers say that pumping doesn't “empty” the breast as efficiently as their baby. Often after pumping, some mums can still express more milk with their hands. Some say that pain, stress, tension and tiredness can affect the hormone release responsible for milk being “let down” and can reduce the amount of milk collected; and
3. Pumping is a poor experience for mothers—surveys of mothers indicate about 85% of mothers say that pumping makes them “feel like a cow”. Others say that it feels overly clinical and they feel isolated

Mothers tend to put up with this experience because pumps allow them to feed breast milk to babies who struggle to feed such as premature babies or babies with tongue ties. Pumps also allow mothers to store milk, increase supply and relieve milk duct blockages and even allow mothers to return to work and continue to supply breast milk to their baby.

However, there is an ongoing need to improve the experience that mothers have with the current milk collection process, by reducing pain and discomfort. It would be advantageous to improve efficiency of milk expression (i.e., collecting more milk in less time) and providing an experience more akin to a baby than prior art devices.

In view of the above, it is desirable to have a device or insert for a breast pump that addresses and ameliorates at least one or more of the prior art deficiencies or at least provides a practical variation to avert from one of more of the prior art deficiencies.

It is to be understood that, if any prior art information is referred to herein, such reference does not constitute an admission that the information forms part of the common general knowledge in the art, in Australia or any other country.

### SUMMARY OF INVENTION

According to a first aspect of the present invention, there is provided a breast pump funnel integral or engageable with a breast pump providing intermittent pumping for drawing breast milk into a connected container, the breast pump funnel having: a) an outer frame, b) an inner malleable liner located at least partially within the outer frame and in use forming a contact surface to the breast; wherein the outer frame, the inner malleable liner or both form a continuous restricting funnel for receiving an intermittent negative pressure pumping.

The breast pump funnel of the present invention not only makes pumping more comfortable for women experiencing pain, it decreases swelling and chaffing and approximates the suckling effect of a real baby.

Preferably, at least the inner malleable liner of the breast pump changes shape on each cycle of the intermittent negative pressure pumping wherein a suckling effect occurs. This provides a pumping technology that mimics a baby's natural sucking rhythm with a patterned intermittent pumping.

The inner malleable liner can have a shaped back engaging the outer frame at a pivot and on each cycle of the intermittent negative pressure pumping the inner malleable liner rocks on the pivot.

The shaped back preferably includes: a plurality of concentric resilient compressible rings contacting with the outer frame; at least one transition structure located between a first and second funnel contact ring, wherein the second funnel contact ring acts as a pivot on a contact surface portion of the outer frame by the first transition structure; and wherein under negative pressure the second funnel contact ring pivots allowing resilient deformable movement of the first funnel contact ring substantially in unison with the breast and nipple at the throat to minimize contact between the nipple and the tube portion of the breast cap.

The outer frame can have a continuous shape. Preferably, the outer frame is a restrictive shape forming a base for the inner malleable liner. The restrictive shape can include a ring of straps. The restrictive shape can include a fixed shaped funnel. The restrictive shape can include a fixed shaped funnel. The fixed shape funnel forming the restrictive shape can include a frusto-conical fixed shape.

In a related aspect of the invention there is disclosed a breast pump insert adapted to fit onto a fixed shaped funnel portion of a breast cap used in a vacuum generating breast pump, the breast pump insert comprising: an inner malleable liner having: a breast receiving side including a mouth having an open end shaped to receive and contact the areola of a woman's breast and a throat of reduced size to receive the woman's nipple; and a shaped back opposite the breast receiving side for mounting the funnel portion of the cap, the shaped back and breast receiving side separated by an edge, wherein the shaped back having a plurality of concentric resilient compressible funnel contact rings formed by the edge adapted to mount the funnel portion of the breast cap in a sealing condition, wherein a first funnel contact ring is located about the throat and is adapted to contact an inner surface portion of the funnel portion of the breast cap adjacent to and above a tube portion of the breast cap; at least one transition structure in the edge located between the first funnel contact ring and a second funnel contact ring upstream from the throat; wherein the second funnel contact ring acts as a pivot on a surface contact portion of the funnel portion by the first transition structure; and wherein under negative pressure the second funnel contact ring pivots on the surface contact portion of the funnel portion allowing resilient deformable movement of the first funnel contact ring substantially in unison with the breast and nipple at the throat to minimize contact between the nipple and the tube portion of the breast cap.

The first resilient funnel contact ring can include an inner compression ring and the second resilient funnel contact ring can include an outer compression ring, wherein the at least one transition structure therebetween can comprise at least a first and second curved transitions to provide structural continuity between the outer compression ring and the inner compression ring.

Preferably, the first curved transition of the at least one transition structure is formed by an outer edge of the inner compression ring which is turned back in on itself, wherein the first curved transition is effective to create sufficient resilience to the insert to substantially maintain a circular or rounded throat when subjected to negative pressure.

The breast insert can comprise a third circumferential funnel contact ring forming a lip for connecting the mouth to an external surface portion of the breast pump funnel portion.

In one preferred embodiment, the lip folds back on itself to form a funnel edge recess adapted to receive an edge portion of the funnel therein, and wherein the third circumferential funnel contact ring is adapted to contact an external surface portion of the funnel to form a pressure tight seal therewith. Preferably, the funnel edge recess is dimensioned to create an airtight seal between the lip and the funnel of the breast cap.

The first, second and third concentric funnel contact rings can include a funnel contact surface portion in substantial alignment with each other for contacting the inner and outer surfaces of the funnel of the breast cap in a sealable condition for effective suction.

The breast pump insert can further include a second transition structure in the edge between the outer compression ring and the third circumferential funnel contact ring. The second transition structure can include a curved transition adjacent the outer compression ring which provides structural continuity between the funnel edge recess and the outer compression ring.

Preferably, the extent of deformation of the inner compression ring is limited such that the diameter of the throat is constricted sufficiently to maintain a light compressive pressure on the nipple and in turn to maintain a seal with the ‘Breast Pump Insert’, the nipple and breast, and the breast pump funnel.

Preferably the mouth of the breast pump insert comprises a cushion surface effective for contact with human skin.

In a related aspect of the invention there is disclosed an insert adapted to fit onto a rigid funnel portion of a vacuum generating breast pump, the breast pump insert comprising: an outer surface portion including a mouth portion having an open end shaped to receive and contact the areola of a woman's breast and a nipple hole formed by an inner portion of reduced size to receive the woman's nipple; a funnel contact portion opposite the outer surface portion and separated by an edge, the edge including a plurality of concentric funnel contact rings adapted to mount the funnel portion, wherein at least a first ring is formed about the nipple hole and is adapted to contact an inner surface portion of the funnel portion adjacent to and above a funnel tube; at least a first transition structure between the first ring and a second ring; wherein the second ring creates a pivot allowing resilient movement of the first ring relative thereto to compliantly deform under negative pressure in unison with the breast and nipple while distributing pressure to evenly support the mouth.

In a further related aspect of the invention there is disclosed an insert adapted to fit into a funnel portion of a vacuum generating breast pump comprising: a mouth portion having an open end shaped to receive and contact the areola of a woman's breast and an inner portion of reduced size to receive the woman's nipple, a circumferential lip for connecting the mouth portion to the breast pump funnel portion to form a pressure tight seal therewith, the mouth portion having a wall with an inner surface adapted to contact the woman's areola around the nipple and an opposite (outer) surface having a first (inner) and second (outer) concentric compression rings, wherein the outer compression ring is intermediate the inner compression ring and circumferential lip; a first (inner) curved transition recess between the inner and outer compression rings; a second (outer) curved transition recess between the outer compression ring and the lip; and a recess between the mouth and the lip for receiving an upper end portion of the funnel; wherein the lip and first and second compression rings are substantially aligned such that each contacts an inner surface of the funnel portion in operation substantially reducing deformation of the inner compression ring concomitantly reducing friction on the breast and nipple when a negative pressure is applied by the breast pump.

In yet a further related aspect of the invention there is disclosed a breast insert adapted to fit into a rigid funnel portion of a breast cap of a vacuum generating breast pump, comprising: a mouth portion having a wall comprising: an inner surface shaped to receive and contact the areola of a woman's breast and an inner throat portion of reduced size to receive the woman's nipple; and an outer surface having at least three concentric compression rings, a first of the concentric compression rings forming an inner compression ring adjacent the throat; a second of said rings forming a circumferential lip adjacent the mouth, wherein the lip folds back on itself to form a funnel edge recess between the mouth and the lip adapted to receive an edge portion of the rigid funnel therein and wherein in a fitted condition the lip engages an external surface of the rigid funnel; and the third of said rings being an outer compression ring intermediate the inner ring and lip; a first curved transition structure between the inner and outer compression rings; a second curved transition recess between the outer compression ring and the lip; wherein the location of the inner compression ring position within the funnel relative to the lip of the funnel substantially prevents the inner compression ring from entering the funnel tube

In a further related aspect of the invention there is disclosed a method of improving collection of breastmilk including: providing a breast insert comprising: a mouth portion having an open end shaped to receive and contact the areola of a woman's breast and an inner portion of reduced size to receive the woman's nipple, a circumferential lip for connecting the mouth portion to the breast pump funnel portion to form a pressure tight seal therewith, the mouth portion having a wall with an inner surface adapted to contact the woman's areola around the nipple and an opposite (outer) surface having a first (inner) and second (outer) concentric compression rings, wherein the outer compression ring is intermediate the inner compression ring and circumferential lip; a first (inner) curved transition structure between the inner and outer compression rings; a second (outer) curved transition structure between the outer compression ring and the lip; and a recess between the mouth and the lip for receiving an upper end portion of the funnel; wherein the lip and first and second compression rings are substantially aligned; inserting the breast insert into the funnel such that the concentric rings contact an inner and outer surface of the funnel portion in a sealing engagement; applying negative pressure to the rigid funnel; wherein the outer compression ring pivots at the contact surface with the funnel and the inner compression ring deforms beyond its resting contact point with the funnel progressively decreasing the diameter at the throat without entering the tube portion.

In operation, as negative vacuum is applied the inner compression ring is displaced resulting in progressively decreasing the diameter of the throat where the nipple is located; and wherein the change at the inner compression ring allows compliant deformation of the inner compression ring at or close to the throat sufficient to compliment movement of the breast under a vacuum so as to minimize abrasion of the nipple with the tube portion of the funnel, which would otherwise cause rubbing of the nipple on the funnel tube, and the deformation of the inner ring approximates a suckling reflex of an infant which promotes milk production.

In a related aspect of the invention there is disclosed a breast pump insert adapted to fit onto a funnel portion of a breast cap used in a vacuum generating breast pump, the breast pump insert comprising: a breast receiving side including a mouth having an open end shaped to receive and contact the areola of a woman's breast and a throat of reduced size to receive the woman's nipple; a funnel mounting side opposite the breast receiving side separated by an edge, the funnel mounting side having a plurality of concentric resilient compressible funnel contact rings formed by the edge adapted to mount the rigid funnel portion of the breast cap in a sealing condition, wherein a first funnel contact ring is located about the throat and is adapted to contact an inner surface portion of the rigid funnel portion of the breast cap adjacent to and above a tube portion of the breast cap; at least one transition structure in the edge located between the first funnel contact ring and a second funnel contact ring upstream from the throat; wherein the second funnel contact ring acts as a pivot on an inner surface portion of the rigid funnel portion by the first transition structure, and wherein under negative pressure the second funnel contact ring pivots allowing resilient deformable movement of the first funnel contact ring substantially in unison with the breast and nipple at the throat to minimize contact between the nipple and the rigid funnel tube of the breast cap.

The insert of the present invention represents an improvement in the experience of using a breast pump because the deformation of the structure at the throat region of the insert under negative pressure more closely characterizes the physical suckling of a baby, and whereas rubbing ordinarily occurs between the nipple and the rigid tube portion of the breast cap, the deformation of the first funnel contact ring at the throat substantially avoids the nipple from being sucked into the breast cap funnel tube and therefore from abrasion or rubbing on the rigid funnel tube surfaces which is the cause of significant pain and discomfort associated with prior art devices.

Compared to the prior art, there is less pain associated with expressing milk with improved efficiency of milk flow. The substantially corresponding deformation between the breast pump insert with the breast and nipple referred to a compliant deformation provides a significantly more comfortable surface and therefore experience for mothers of newborns.

Preferably the first resilient funnel contact ring is an inner compression ring and the second resilient funnel contact ring is an outer compression ring, wherein the at least one transition structure therebetween comprises at least a first and second curved transitions to provide structural continuity between the outer compression ring and the inner compression ring.

Preferably the first curved transition of the at least one transition structure is formed by an outer edge of the inner compression ring which is turned back in on itself, wherein the first curved transition is effective to create sufficient resilience to the insert to substantially maintain a circular or rounded throat when subjected to negative pressure.

Preferably the breast insert comprises a third circumferential funnel contact ring forming a lip for connecting the mouth to an external surface portion of the breast pump funnel portion. Preferably the lip folds back on itself to form a funnel edge recess adapted to receive an edge portion of the rigid funnel therein, and wherein the third circumferential funnel contact ring is adapted to contact an external surface portion of the funnel to form a pressure tight seal therewith.

Preferably the funnel edge recess is dimensioned to create a airtight seal between the lip and the funnel of the breast cap.

Preferably the first, second and third concentric funnel contact rings include a funnel contact surface portion in substantial alignment with each other for contacting the inner and outer surfaces of the funnel of the breast cap in a sealable condition for effective suction.

Preferably the insert can include a second transition structure in the edge between the outer compression ring and the third circumferential funnel contact ring.

Preferably the second transition structure includes a curved transition adjacent the outer compression ring which provides structural continuity between the funnel edge recess and the outer compression ring.

Preferably, the extent of deformation of the inner compression ring is limited such that the diameter of the throat is reduced sufficiently to maintain a light compressive pressure on the nipple and in turn to maintain a seal with the ‘Breast Pump Insert’, the nipple and breast, and the breast pump funnel.

Preferably the mouth comprises a cushion surface effective for contact with human skin. In one preferred embodiment of the present invention, the insert includes a funnel portion integrally molded to the funnel mounting side thereof forming a single piece insert so that the insert can be directly attached to a tube portion of a breast cap.

Without being bound by theory, the features of the inner compression ring position within the funnel relative to the lip of the funnel, transition structure between the inner and outer compression rings together with the outer compression ring pivot, combine to constrict the throat and simultaneously avoid the inner compression ring from entering the tube portion of the breast cap under vacuum. As deformation of the inner compression ring occurs with pivoting motion of the outer compression ring under vacuum, the inner compression ring compliantly deforms in unison with the breast and nipple. The constriction at the throat caused by deformation movement of the inner compression ring and matched deformation between the breast pump insert with the breast and nipple provide greater support and a more comfortable surface.

In a related aspect of the invention there is provided a breast insert adapted to fit into a funnel portion of a breast cap of a vacuum generating breast pump, comprising: a mouth portion having a wall comprising: an inner surface shaped to receive and contact the areola of a woman's breast and an inner throat portion of reduced size to receive the woman's nipple; and an outer surface having at least three concentric compression rings, a first of the concentric compression rings forming an inner compression ring adjacent the throat; a second of said rings forming a circumferential lip adjacent the mouth, wherein the lip folds back on itself to form a funnel edge recess between the mouth and the lip adapted to receive an edge portion of the funnel therein and wherein in a fitted condition the lip engages an external surface of the funnel; and the third of said rings being an outer compression ring intermediate the inner ring and lip; a first curved transition structure between the inner and outer compression rings; a second structural transition recess between the outer compression ring and the lip; wherein the location of the inner compression ring within the funnel relative to the lip of the funnel substantially prevents the inner compression ring from entering the tube portion of the breast cap.

The throat portion of the insert for receiving the nipple can be sized to match different nipple diameters.

Preferably, the funnel edge recess has an overlap and opening width suitable to create a good seal between the lip and the funnel.

Preferably the second structural transition recess structure can include a curved transition shaped contour adjacent the outer compression ring which provides structural continuity between the funnel edge recess and the outer compression ring.

Preferably the first transition structure between the inner and outer compression rings comprises a first and second curved transitions. The curved transitions provide structural continuity between the outer compression ring and the inner compression ring.

Preferably the inner compression ring is configured to create enough material stiffness to maintain a circular or rounded opening to support the throat to substantially prevent collapsing under vacuum.

The inner compression ring stretches and deforms according to pressure variations exerted from the breast pump or from the breast and nipple exerting pressure on the wall of the throat.

The outer compression ring creates a pivot for the inner compression ring to compliantly deform in unison with the breast and nipple to constrict the throat diameter while distributing pressure to evenly support the mouth.

The inner compression ring maintains light compressive pressure on the nipple to maintain a seal with the ‘Breast Pump Insert’, the nipple and breast, and the breast pump funnel

In a related aspect of the invention there is provided a method of improving collection of breastmilk including: providing a breast insert comprising: a mouth portion having an open end shaped to receive and contact the areola of a woman's breast and an inner portion of reduced size to receive the woman's nipple, a circumferential lip for connecting the mouth portion to the breast pump funnel portion to form a pressure tight seal therewith, the mouth portion having a wall with an inner surface adapted to contact the woman's areola around the nipple and an opposite (outer) surface having a first (inner) and second (outer) concentric compression rings, wherein the outer compression ring is intermediate the inner compression ring and circumferential lip; a first (inner) curved transition recess between the inner and outer compression rings; a second (outer) curved transition recess between the outer compression ring and the lip; and a recess between the mouth and the lip for receiving an upper end portion of the funnel; wherein the lip and first and second compression rings are substantially aligned; inserting the breast insert into the funnel such that the concentric rings contact an inner and outer surface of the funnel portion in a sealing engagement; applying negative pressure to the funnel; wherein the outer compression ring pivots at the contact surface with the funnel and the inner compression ring deforms beyond its resting contact point with the funnel progressively decreasing the diameter at the throat without entering the tube portion.

As the vacuum is applied, the inner compression ring is displaced resulting in progressively decreasing the diameter of the throat where the nipple is located. This change at the inner compression ring allows compliant deformation of the inner compression ring at or close to the throat sufficient to compliment movement of the breast under a vacuum so as to minimize abrasion of the nipple with the tube portion of the breast cap, which would otherwise cause rubbing of the nipple on the tube portion, and the deformation of the inner ring approximates a suckling reflex of an infant which promotes milk production.

### Benefits of the Invention Include

The insert directly stimulates the nerve endings in the areola of the breast. During lactation, the effective removal of the milk from the breast happens as a result of the milking action provided by the baby's mouth and jaw movements once the “let down” reflex is activated due to the mother's “oxytocin” release.

Nipple hole fits different nipple diameters.

The inner compression ring prevents the nipple from being sucked into the breast pump funnel tube preventing the nipple from rubbing on the rigid breast pump funnel tube surfaces.

The constriction of the throat by deformation of the inner compression ring under vacuum approximates a suckling reflex, which encourages a physiological response to promote greater milk production.

The combined concentric compression rings can create a seal on multiple common breast pump funnels.

The curved transitions reduce the risk of tearing and fatigue than angular forms.

The curved transition structures are easy to clean compared with angular forms.

The outer lip of the inner compression ring provides support for the nipple, cushioning and preventing the nipple from rubbing on the rigid breast pump funnel tube surfaces

The airtight seal across the ‘Breast Pump Insert’, the nipple and breast, and the breast pump funnel prevents loss of breast milk

The compliant deformation of the nipple hole membrane about the outer compression ring reduces friction on the breast and nipple when in contact with the ‘Breast Pump Insert’

Other aspects of the invention are also disclosed with reference to accompanying drawings and examples.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is a cross-section schematic representation of a breast pump funnel integral or engageable with a breast pump in accordance with a preferred embodiment of the present invention;

FIG. 2 is a cross-sectional perspective schematic representation of the breast pump funnel integral or engageable with a breast pump of FIG. 1;

FIG. 3 is a cross-section schematic representation of a breast insert in accordance with a preferred embodiment of the present invention positioned in a rigid funnel of a breast cap;

FIG. 4 is a cross-sectional perspective schematic representation of the breast insert of FIG. 3 positioned in a rigid funnel of a breast cap;

FIG. 5 is a close up schematic representation of a part of the breast insert shown in FIG. 4;

FIG. 6 is a cross-section schematic representation of a breast insert in accordance with an alternate embodiment of the present invention;

FIG. 7 is a schematic representation of the breast insert of FIG. 6 in a side elevation;

FIG. 8 is a schematic representation of the breast insert of FIGS. 6 and 7 in overhead plan view;

FIG. 9 is a cross-section schematic representation of a breast insert in accordance with a further alternate embodiment of the present invention with breast cap in dotted outline;

FIG. 10 is a schematic representation of the breast insert of FIG. 9 in a side elevation;

FIG. 11 is a schematic representation of the breast insert of FIGS. 9 and 10 in overhead plan view;

FIG. 12 is a cross-section schematic representation of a breast insert in accordance with a further alternate embodiment of the present invention with breast cap in dotted outline;

FIG. 13 is a schematic representation of the breast insert of FIG. 12 in a side elevation;

FIG. 14 is a schematic representation of the breast insert of FIGS. 12 and 13 in overhead plan view;

FIG. 15 is a cross-section schematic representation of a breast insert in accordance with a further embodiment of the present invention;

FIG. 16 is a schematic representation of the breast insert of FIG. 15 in a side elevation;

FIG. 17 is a schematic representation of the breast insert of FIGS. 15 and 16 in overhead plan view;

FIG. 18 is a cross-section schematic representation of a breast insert in accordance with a further embodiment of the present invention;

FIG. 19 is a schematic representation of the breast insert of FIG. 18 in a side elevation;

FIG. 20 is a schematic representation of the breast insert of FIGS. 18 and 19 in overhead plan view;

FIG. 21 is a cross-section schematic representation of a breast insert in accordance with a further embodiment of the present invention;

FIG. 22 is a schematic representation of the breast insert of FIG. 21 in a side elevation;

FIG. 23 is a schematic representation of the breast insert of FIGS. 21 and 22 in overhead plan view;

FIG. 24 is a cross-section schematic representation of a breast insert in accordance with a further preferred embodiment of the present invention positioned in a rigid funnel in a static condition;

FIG. 25 is a cross-section schematic representation of the breast insert in FIG. 24 positioned in a rigid funnel of a breast cap, showing static position in dotted outline, under application of negative pressure;

FIG. 26 is a cross-section schematic representation of a breast insert in accordance with a further embodiment of the present invention;

FIG. 27 is a cross-section schematic representation of the breast insert of FIG. 26 in a rigid funnel in a static condition;

FIG. 28 is a cross-section schematic representation of the breast insert in FIG. 27 positioned in a rigid funnel of a breast cap, showing static position in dotted outline, under application of negative pressure;

FIG. 29 shows results of study participants characteristics—number of children, pump type and use;

FIG. 30 shows results of study participants characteristics—Comfort rating of pumping with a breast pump insert of the present invention attached (out of 10);

FIG. 31 shows results of study participants characteristics—Change in median scores of nipple swelling, nipple redness/colour change, broken skin/chaffing and comfort;

FIG. 32 shows results of study participants characteristics—Change in ‘feel’ of the cushion, starting from 5/10 setpoint;

FIG. 33 shows results of study participants characteristics—Milk Collected

### DESCRIPTION OF PREFERRED EMBODIMENTS

It should be noted in the following description that like or the same reference numerals in different embodiments denote the same or similar features.

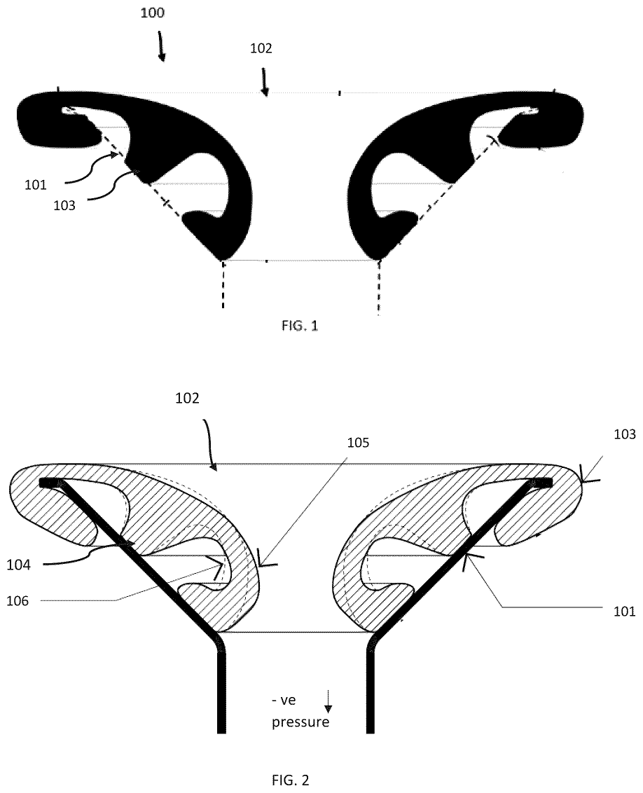

Referring to FIG. 1 there is shown a breast pump funnel 100 integral or engageable with a breast pump (not shown) providing intermittent pumping for drawing breast milk into a connected container, the breast pump funnel having an outer frame 101, an inner malleable liner 102 located at least partially within the outer frame and in use forming a contact surface to the breast; wherein the outer frame 101, the inner malleable liner 102 or both form a continuous restricting funnel for receiving an intermittent negative pressure pumping by the breast pump. The inner malleable liner of the breast pump is adapted to change shape on each cycle of the intermittent negative pressure pumping wherein a suckling effect occurs

As shown, the inner malleable liner 102 has a shaped back 103 engaging the outer frame 101 at a pivot 104. On each cycle of the intermittent negative pressure pumping by the breast pump, the inner malleable liner rocks on the pivot 104. This is shown in FIG. 2 where continuous lines 105 show deformation under negative pressure from a normal position (shown in dashed lines 106). In an operating condition, under negative pressure, the shaped back of the inner malleable liner 102 pivots allowing resilient deformable movement of the breast pump funnel substantially in unison with the breast and nipple. This improves comfort and provides a close physical approximation to a suckling baby.

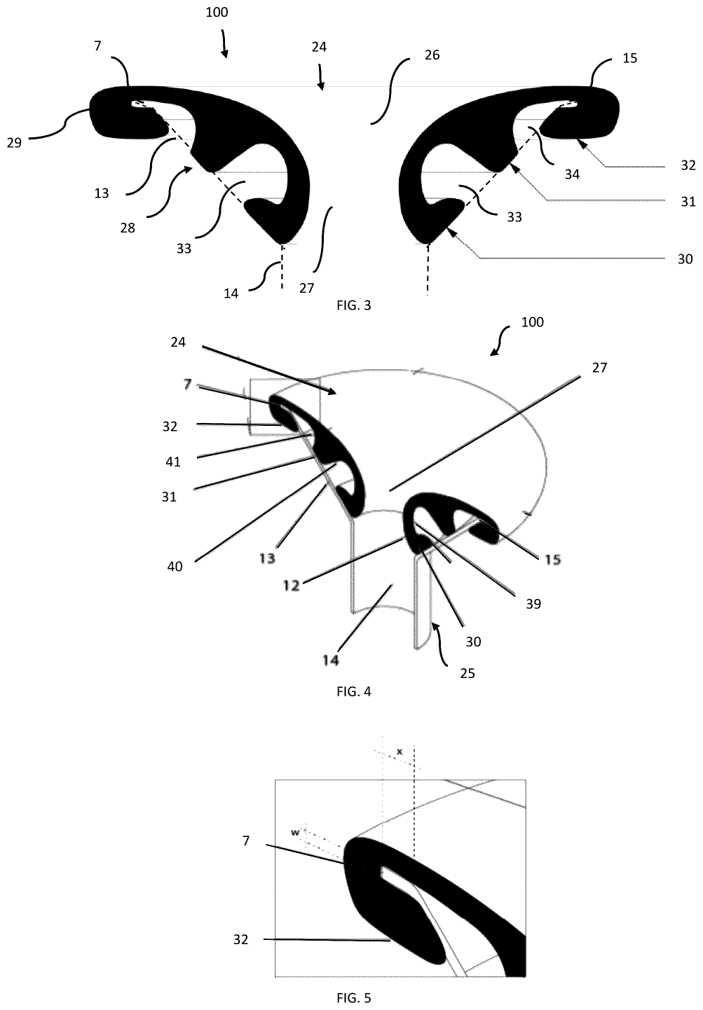

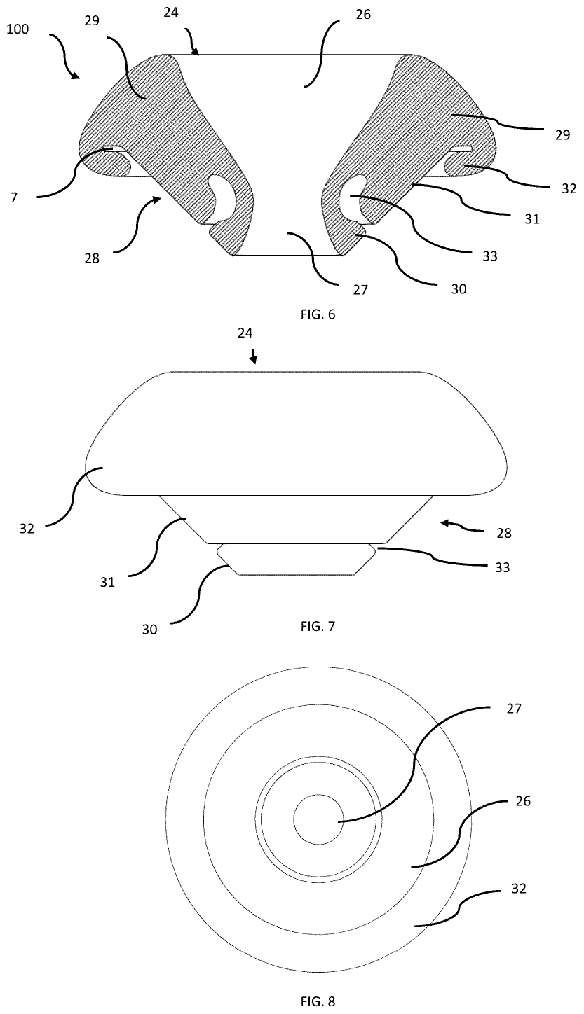

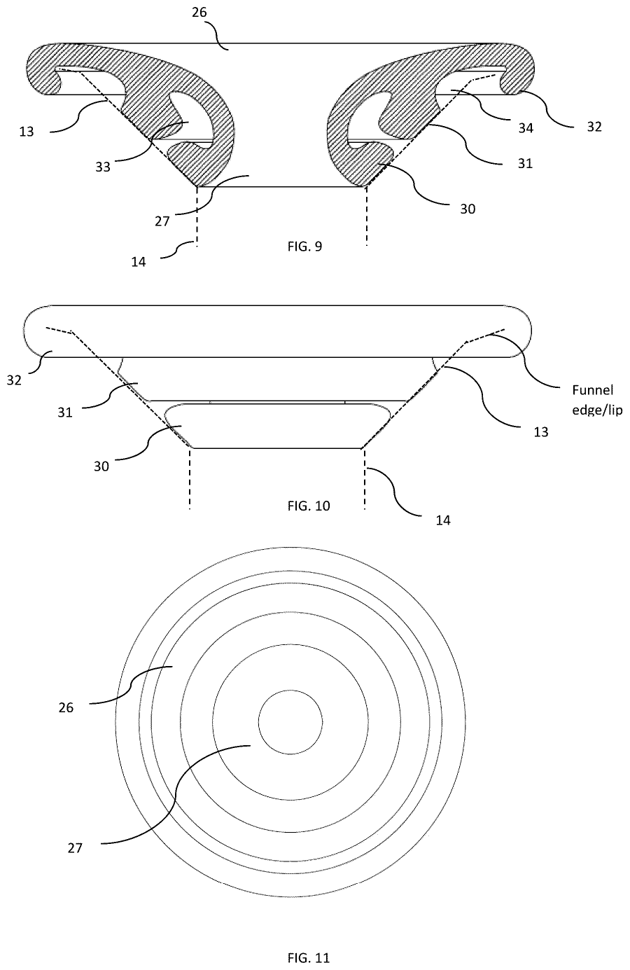

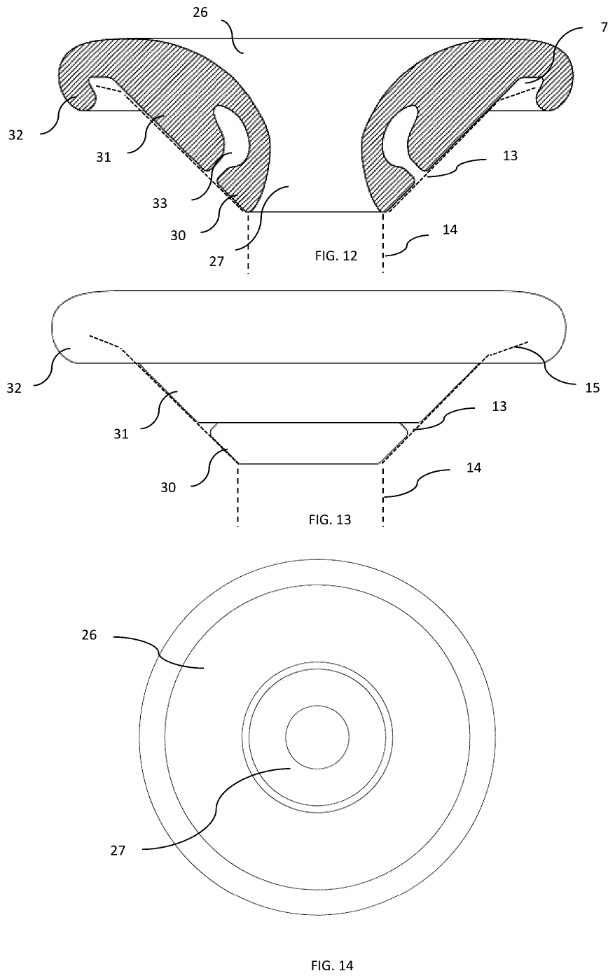

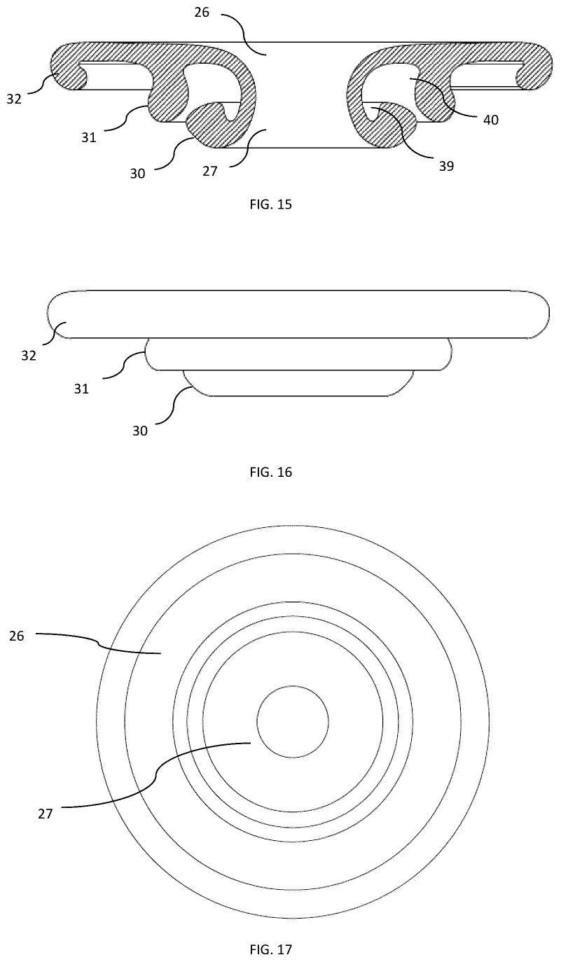

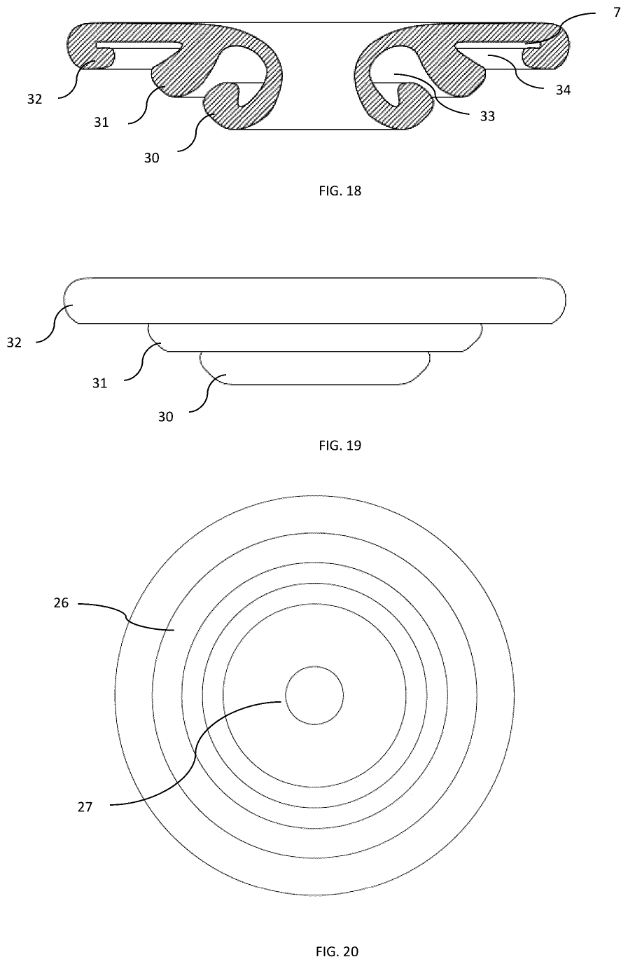

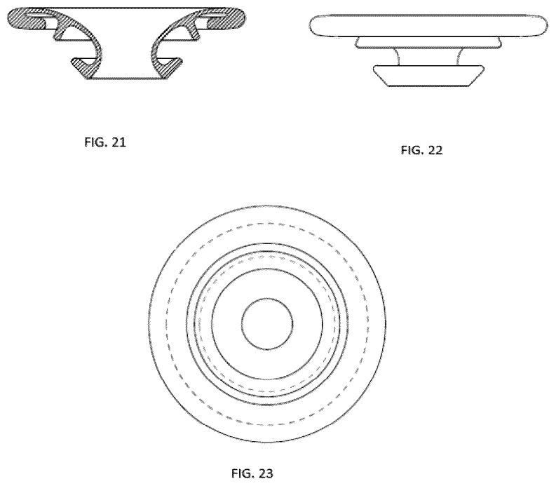

Referring to FIGS. 3 to 20, there is shown a breast pump insert 100 adapted to fit into an outer frame such as a funnel portion 13 of a breast cap 25 used in a vacuum generating breast pump (not shown) to approximate the suckling reflex of a baby and to allow improved cushioning to alleviate pain otherwise commonly associated with using a breast pump.

The breast pump insert 100 comprises an inner malleable liner having a shaped back including a plurality of concentric resilient compressible rings contacting with the funnel portion 13 of the outer frame 25. When a negative pressure is applied, at least the inner malleable liner compresses and deforms creating a suckling effect on each cycle of the intermittent negative pressure pumping.

The breast pump insert includes breast receiving side 24 including a mouth 26 having an open end shaped to receive and contact the areola of a woman's breast and a throat 27 sized to receive the woman's nipple; and a funnel mounting side 28 opposite the breast receiving side separated by an edge 29. In FIG. 3, the funnel mounting side 28 includes three concentric resilient funnel contact rings 30, 31 and 32 formed by the edge 29. As best shown in FIGS. 3, 4, and 8 to 12, the resilient funnel contact rings 30, 31 and 32 are in substantial alignment to mount opposite sides of the funnel portion 13 in a sealing engagement. Sealing engagement is shown by ring 32 contacting and extending circumferentially about an external surface of the funnel 13 and both resilient funnel contact rings 30 and 31 contacting and extending circumferentially about an internal surface of the funnel 13 in spaced apart relation.

As shown in the figures, the resilient funnel contact ring 30 is an inner compression ring formed about the throat 27 and is adapted to contact an inside surface portion of the funnel portion 13 of the breast cap or outer frame 25 adjacent to and above the tube portion 14 of the breast cap. The resilient funnel contact ring 31 is an outer compression ring adapted to contact an inside surface portion of the funnel portion 13 spaced from the inner compression ring. In one embodiment the inner malleable liner is fabricated from a soft silicone.

The figures each show at least one transition structure 33 in the edge 29 located between the inner compression ring 30 and the outer compression ring 31. The configuration of the first transition structure 33 includes an inner curved transition surface 39 adjacent the inner compression ring 30 and an outer curved transition surface 40 close to the outer compression ring 31.

The at least one transition structure 33 is configured by the inner and outer curved transition surfaces 39 and 40 such that the outer compression ring 31 acts as a pivot point on the funnel surface.

FIGS. 5, 6, 11 17, 20, 23, and 24 show a second structural transition recess 34 between the outer compression ring 31 and the resilient funnel contact ring 32. In this embodiment the second structural transition recess includes an inner curved transition surface 41 which structurally supports the pivot action of the outer compression ring 31 on the surface of the funnel which it is in contact with.

As shown in close up view in FIG. 5, the resilient funnel contact ring 32 turns back on itself forming a funnel edge recess 7. The recess has a defined depth ‘x’ and width ‘w’ configured to receive a funnel lip 15 of the rigid funnel portion 13 therein. The resilient funnel contact ring 32 is further configured to align with the inner and outer compression rings 30 and 31 so that ring 32 makes contact with an outside surface of the funnel portion 13 while the inner and outer compression rings contact an inside surface portion of the funnel providing an air tight seal.

The breast insert can be made of soft materials such as silicones which possess physical characteristics including compressibility and malleability without causing any harmful allergic reactions by contact.

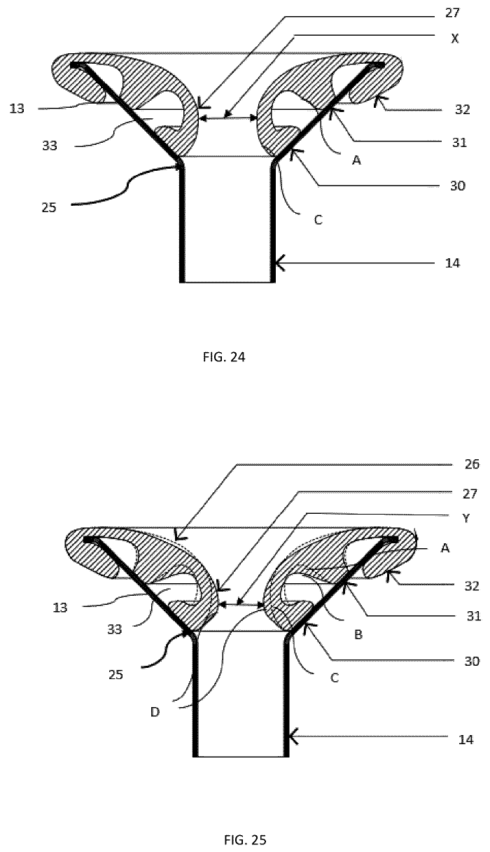

FIGS. 24 and 25 and FIGS. 27 and 28 show the effect of the insert 100 under applied vacuum, where the diameter of the throat 27 progressively decreases with deformation of the inner compression ring 30. FIG. 24 shows the breast pump insert positioned in sealing engagement on the funnel prior to application of a vacuum where points A and C represent normal positions of the inner and outer compression rings 30 and 31 respectively and where the throat 27 has a resting diameter ‘X’.

In an operating condition (see FIG. 25) under a negative pressure (vacuum), the outer compression ring 31 pivots at the contact surface with the funnel 13 (shown by “B”) and the inner compression ring deforms to an extent shown by ‘D’ beyond its resting contact point with the funnel progressively decreasing the diameter at the throat represented by ‘Y’. As the vacuum is applied, the inner compression ring is displaced resulting in progressively decreasing the diameter of the throat where the nipple is located. This change at the inner compression ring allows compliant deformation of the inner compression ring at or close to the throat 27 sufficient to compliment movement of the breast under a vacuum so as to minimize abrasion of the nipple with the tube portion 13 of the breast cap 25, which would otherwise cause rubbing of the nipple on the tube portion, and the deformation of the inner ring approximates a suckling reflex of an infant which promotes milk production.

The ability of the second funnel contact ring acting as a pivot on a surface portion of the funnel portion as provided by the first transition structure, allows for the resilient movement of the inner ring and contraction of the throat relative thereto to compliantly deform under negative pressure substantially in unison with the breast and nipple while distributing pressure to evenly support the mouth portion of the breast pump insert.

### Example 1— Comfort Analysis

Method

28 participants were used taken from a local community mothers' group targeting a population of lactating women using a range of conventional breast pumps. The participants were divided into two groups based on their response to the question “How comfortable is your current pump?”. Cohort A: scores of 5 or more out of 10 (i.e. relatively comfortable), n=15. Cohort B: scores of less than 5 out of 10 (i.e. uncomfortable), n=13.

Each participant completed a pre-pump survey (n=28), tried the breast pump insert according to an embodiment of the present invention on their pump and then completed a post-pump survey (n=26, 2 did not complete). Most participants also completed a follow-up interview.

The surveys were structured to gather information in five areas:
1. Background information on breast pump use of participant
2. Comfort
3. Nipple redness, swelling, chaffing/broken skin
4. Feel Volumes.

The surveys included both quantitative and qualitative questions.

### Results

Observations on Breast Pump Use

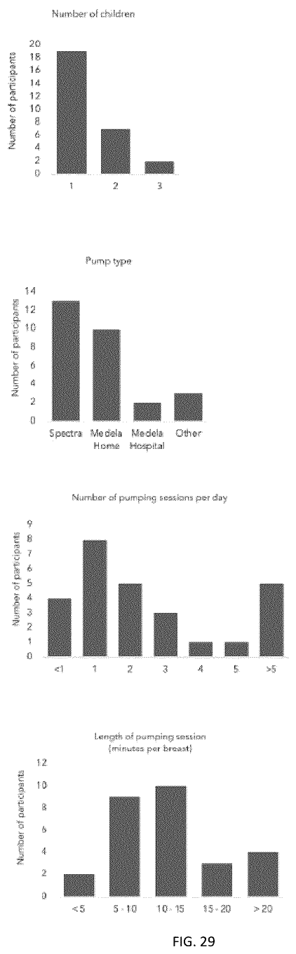

FIG. 29 describes the distribution of participants across number of children, pump type, pumping sessions per day and session time per pump.

Observations on comfort and nipple swelling, redness and chaffing.

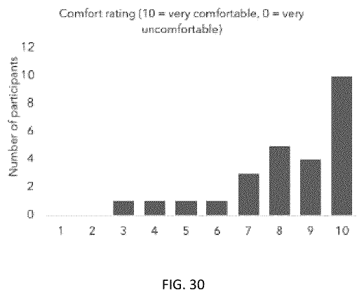

FIG. 30 shows the distribution of comfort ranking (out of 10) with the cushion attached. Note that instant ‘comfort’ rating is inverse to ‘pain’ ratings where represents “very comfortable” and 0 represents “very uncomfortable”. Our swelling, redness and chaffing ratings are more typical, with 10 representing “severe” and 0 representing “none at all”.

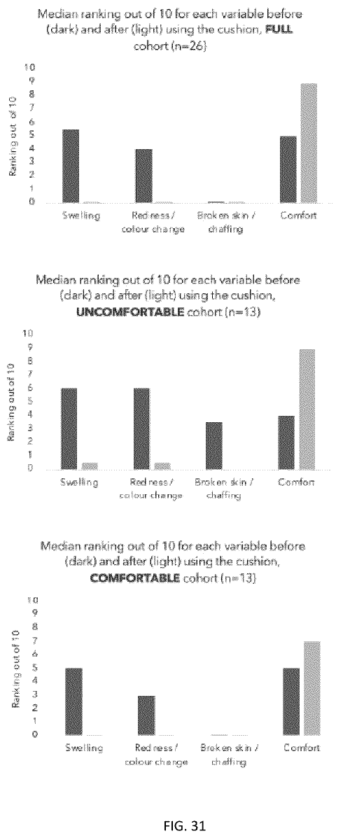

FIG. 31 shows the change in median scores of nipple swelling, nipple redness/colour change, broken skin/chaffing and comfort, from using the pump without the cushion (dark) to using the pump with the cushion (light). The first chart shows all participants, the second and third charts show the results for each cohort: uncomfortable (comfort score starting at less than 5 out of 10) and comfortable (comfort score starting at more than 5 out of 10).

The following changes were observed:
increased comfort (median score: 5/10 to 9/10 for all cohort)
relief from swelling (median score: 6/10 to 0/10)
relief from redness and colour change (median score: 4/10 to 0/10)
relief from broken skin and chaffing (4/10 to 0/10) for cohort of women self-describing as finding pumping uncomfortable without the cushion.

Observations on Cushion ‘Feel’

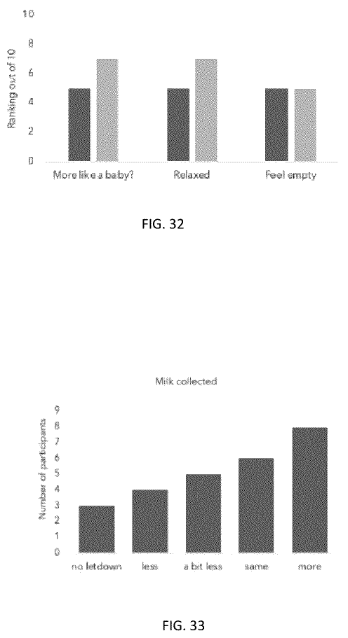

FIG. 32 shows the change in ‘feel’ of the cushion, starting from a set base of ‘5’ for each variable. Participants recorded: • feeling more ‘like a baby’ (median score: 5/10 to 7/10) • feeling more relaxed (median score: 5/10 to 7/10) • feeling ‘empty’ (median score: unchanged)

Observations on Milk Volumes Collected

FIG. 33 shows the change in perception of milk volume collected. Volumes weren't measured or tracked before or after; our objective was to identify if there was a major impediment to milk flow rather than identifying incremental and measurable changes in milk volumes. We may investigate this at a later stage. Participants recorded no change in milk volume collected (median score unchanged). Most participants (73%) collected a bit less, same or more milk; however some participants (12%) were unable to gather milk or have a letdown at all.

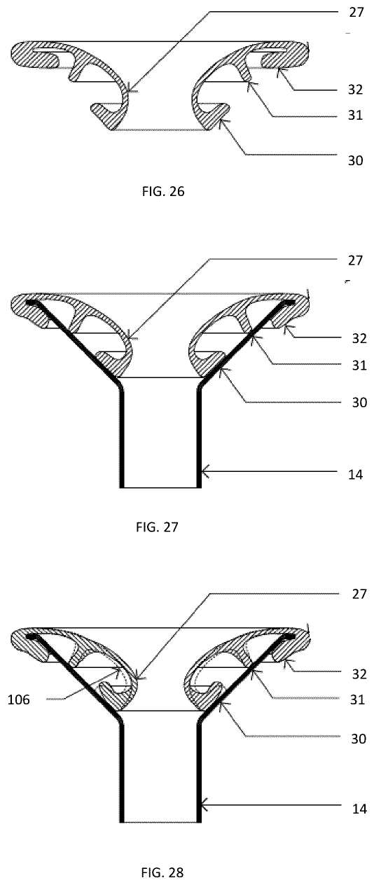

After interviewing these participants, a further breast pump insert according to FIGS. 26 to 28 with less ‘bulk’ was provided that allows for more stimulation at the breast. Preliminary testing, and preliminary results using this embodiment show that 5 out of 6 participants who had struggled with letdown or low volumes were able to collect equivalent or more milk than pumping without the breast pump insert in addition to being more comfortable.

### Benefits of the Invention Include

The inner compression ring prevents the nipple from being sucked into the breast pump tube preventing the nipple from rubbing on the breast pump tube surfaces.

The constriction of the throat by deformation of the inner compression ring under vacuum approximates a suckling reflex, which encourages a physiological response to promote greater milk production.

Greater airtight seal formed between the concentric rings, funnel and breasts/nipple to reduce milk loss.

Interpretation

### EMBODIMENTS

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to a breast insert or cap for a breast pump to improve comfort and milk production efficiency.

1-37. (canceled)

38. A breast pump funnel integral or engageable with a breast pump providing intermittent pumping for drawing breast milk into a connected container, the breast pump funnel comprising:
a) an outer frame;
b) an inner malleable liner located at least partially within the outer frame and configured to form a contact surface to the breast, wherein the inner malleable liner has a shaped back engaging the outer frame at a pivot and on each cycle of the intermittent negative pressure pumping the inner malleable liner rocks on the pivot; and
wherein the outer frame, the inner malleable liner or both form a continuous restricting funnel for receiving an intermittent negative pressure pumping.

39. The breast pump funnel according to claim 38, wherein the shaped back comprises:
a plurality of concentric resilient compressible rings contacting with the outer frame;
at least one transition structure located between a first and second funnel contact ring, wherein the second funnel contact ring acts as a pivot on a contact surface portion of the outer frame by the first transition structure; and
wherein under negative pressure the second funnel contact ring pivots allowing resilient deformable movement of the first funnel contact ring substantially in unison with the breast and nipple at the throat to minimize contact between the nipple and the tube portion of the breast cap.

40. A breast pump insert adapted to fit onto a fixed shaped funnel portion of a breast cap used in a vacuum generating breast pump, the breast pump insert comprising:
an inner malleable liner having:
a breast receiving side including a mouth having an open end shaped to receive and contact the areola of a woman's breast and a throat of reduced size to receive a woman's nipple; and
a shaped back opposite the breast receiving side configured for mounting the funnel portion of the cap, the shaped back and breast receiving side separated by an edge, wherein the shaped back has a plurality of concentric resilient compressible funnel contact rings formed by the edge adapted to mount the funnel portion of the breast cap in a sealing condition, wherein a first funnel contact ring is located about the throat and is adapted to contact an inner surface portion of the funnel portion of the breast cap adjacent to and above a tube portion of the breast cap;
at least one transition structure in the edge located between the first funnel contact ring and a second funnel contact ring upstream from the throat;
wherein the second funnel contact ring acts as a pivot on a surface contact portion of the funnel portion by the first transition structure; and
wherein, under negative pressure, the second funnel contact ring pivots on the surface contact portion of the funnel portion allowing resilient deformable movement of the first funnel contact ring substantially in unison with the breast and nipple at the throat to minimize contact between the nipple and the tube portion of the breast cap.

41. The breast pump insert according to claim 40, wherein the first resilient funnel contact ring is an inner compression ring and the second resilient funnel contact ring is an outer compression ring, and wherein the at least one transition structure therebetween comprises at least a first and second curved transitions to provide structural continuity between the outer compression ring and the inner compression ring.

42. The breast pump insert according to claim 40, wherein the first curved transition of the at least one transition structure is formed by an outer edge of the inner compression ring which is turned back in on itself, wherein the first curved transition is effective to create sufficient resilience to the insert to substantially maintain a circular or rounded throat when subjected to negative pressure.

43. The breast pump insert according to claim 40, wherein the breast insert comprises a third circumferential funnel contact ring forming a lip for connecting the mouth to an external surface portion of the breast pump funnel portion, wherein the lip folds back on itself to form a funnel edge recess adapted to receive an edge portion of the funnel therein, and wherein the third circumferential funnel contact ring is adapted to contact an external surface portion of the funnel to form a pressure tight seal therewith.

44. The breast pump insert according to claim 40, wherein the first, second, and third concentric funnel contact rings include a funnel contact surface portion in substantial alignment with each other for contacting the inner and outer surfaces of the funnel of the breast cap in a sealable condition for effective suction.

45. The breast pump insert according to claim 40, further including a second transition structure in the edge between the outer compression ring and the third circumferential funnel contact ring, wherein the second transition structure includes a curved transition adjacent the outer compression ring which provides structural continuity between the funnel edge recess and the outer compression ring.

46. The breast pump insert according to claim 40, wherein the extent of deformation of the inner compression ring is limited such that the diameter of the throat is constricted sufficiently to maintain a light compressive pressure on the nipple and in turn to maintain a seal with the breast pump insert, the nipple and breast, and the breast pump funnel.

47. The breast pump insert according to claim 40, wherein the mouth comprises a cushion surface effective for contact with human skin.

48. A breast pump insert adapted to fit onto a funnel portion of a breast cap used in a vacuum generating breast pump, the breast pump insert comprising:
a breast receiving side including a mouth having an open end shaped to receive and contact the areola of a woman's breast and a throat of reduced size to receive a woman's nipple;
a funnel mounting side opposite the breast receiving side separated by an edge, the funnel mounting side having a plurality of concentric resilient compressible funnel contact rings formed by the edge adapted to mount the funnel portion of the breast cap in a sealing condition, wherein a first funnel contact ring is located about the throat and is adapted to contact an inner surface portion of the funnel portion of the breast cap adjacent to and above a tube portion of the breast cap;
at least one transition structure in the edge located between the first funnel contact ring and a second funnel contact ring upstream from the throat;
wherein the second funnel contact ring acts as a pivot on an inner surface portion of the funnel portion by the first transition structure, and wherein under negative pressure the second funnel contact ring pivots allowing resilient deformable movement of the first funnel contact ring substantially in unison with the breast and nipple at the throat to minimize contact between the nipple and the tube portion of the breast cap.

49. The breast pump insert according to claim 48, wherein the first resilient funnel contact ring is an inner compression ring and the second resilient funnel contact ring is an outer compression ring, wherein the at least one transition structure therebetween comprises at least a first and second curved transitions to provide structural continuity between the outer compression ring and the inner compression ring.

50. The breast pump insert according to claim 48, wherein the breast insert comprises a third circumferential funnel contact ring forming a lip for connecting the mouth to an external surface portion of the breast pump funnel portion, wherein the lip folds back on itself to form a funnel edge recess adapted to receive an edge portion of the funnel therein, and wherein the third circumferential funnel contact ring is adapted to contact an external surface portion of the funnel to form a pressure tight seal therewith.

51. The breast pump insert according to claim 48, wherein the first, second and third concentric funnel contact rings include a funnel contact surface portion in substantial alignment with each other for contacting the inner and outer surfaces of the funnel of the breast cap in a sealable condition for effective suction.

52. The breast pump insert according to claim 48, further including a second transition structure in the edge between the outer compression ring and the third circumferential funnel contact ring, wherein the second transition structure includes a curved transition adjacent the outer compression ring which provides structural continuity between the funnel edge recess and the outer compression ring.

53. The breast pump insert according to claim 48, wherein the extent of deformation of the inner compression ring is limited such that the diameter of the throat is constricted sufficiently to maintain a light compressive pressure on the nipple and in turn to maintain a seal with the breast pump cap, the nipple and breast.
